(19) United States
(12) Patent Application Publication
(10) Pub. No.: US 2005/0130878 A1
(43) Pub. Date: Jun. 16, 2005

(54) Prevention of photic injury by administering a TACE inhibitor

(75) Inventors: Rouel S. Roque, Fort Worth, TX (US); Bhooma Srinivasan, Irving, TX (US); Robert J. Collier JR., Arlington, TX (US)

Correspondence Address: Teresa J. Schultz;Alcon Research, Ltd., 6201 S. Freeway , Q-148, Fort Worth, TX, 76134-2099, US

(73) Assignees: Alcon, Inc.; University of North Texas Health Science Center

(21) Appl. No.: 10/994,580

(22) Filed: Nov. 22, 2004

Related U.S. Application Data
(60) Provisional application No. 60/525,634, filed on Nov. 26, 2003.

(51) Int. Cl. A61K 38/00

(52) U.S. Cl. 514/2

(57) ABSTRACT

The present invention relates to is directed to methods of preventing neurodegeneration resulting from photo-oxidative stress. The methods of the invention involve inhibiting the action of the tumor necrosis factor-converting enzyme (TACE) in retinal cells that have experienced photic injury.

This application claims priority from U.S. Ser. No. 60/525,634, filed Nov. 26, 2003.

### BACKGROUND OF THE INVENTION

1. Field of the Invention

The present invention relates to the field of retinal degenerative diseases resulting from photic injury. More particularly, the present invention relates to treatment of such diseases by administering to a patient a therapeutically effective amount of a TACE inhibitor.

2. Description of the Related Art

The pathogenesis of retinal degenerative diseases such as age-related macular degeneration (ARMD) and retinitis pigmentosa (RP) is multifaceted and can be triggered by environmental factors in those who are genetically predisposed. One such environmental factor, light exposure, has been identified as a contributing factor to the progression of retinal degenerative disorders such as ARMD (Young 1988). Photo-oxidative stress leading to light damage to retinal cells has been shown to be a useful model for studying retinal degenerative diseases for the following reasons: damage is primarily to the photoreceptors and retinal pigment epithelium of the outer retina (Noell et al. 1966; Bressler et al. 1988; Curcio et al. 1996); they share a common mechanism of cell death, apoptosis (Ge-Zhi et al. 1996; Abler et al. 1996); light has been implicated as an environmental risk factor for progression of ARMD and RP (Taylor et al. 1992; Naash et al. 1996); and therapeutic interventions which inhibit photo-oxidative injury have also been shown to be effective in animal models of heredodegenerative retinal disease (LaVail et al. 1992; Fakforovich et al. 1990).

Photic injury to the retina involves intense light exposure to retinal photoreceptor cells resulting in the activation of the phototransduction cascade leading to cell death via apoptosis (Noell et al. 1966). The extent of the retinal damage is highly dependent on the duration (Moriya et al. 1986) and intensity of light exposure (Organisciak et al. 1998) such that short exposure to bright light or continuous low light exposure for prolonged periods both lead to retinal damage with fewer photoreceptor cells, shorter outer segments, and damaged membranes (Penn et al. 1992). Outer segment changes are the earliest findings from continuous exposure to fluorescent light, whereas exposure to bright light induces changes in the photoreceptor inner segments and pigment epithelial cells before outer segment changes occur (Kuwabara and Gorn 1968).

Although several mechanisms have been proposed to explain the development of apoptotic cell death from photic injury, recent reports showing increased expression of the p75 neurotrophin receptor (p75NTR) in photoreceptor cells in animals with retinal dystrophy (Kahle and Hertel 1992); the survival of photoreceptor cells of p75NTR knock out mice from light exposure as compared with the wild type (Sheedlo et al. 2002); and the anti-p75NTR antibody-mediated delay of light-induced photoreceptor apoptosis in albino Wistar rats suggest that p75NTR may be involved in photoreceptor cell death.

The eye is subjected to continuous light exposure because the primary purpose of the eye is light perception. Therefore, some untreatable diseases and injuries to the eye result from the continuous exposure of the eye to light, coupled with the highly-oxygenated environment in the eye.

The process of light perception is initiated in the photoreceptor cells. The photoreceptor cells are a constituent of the outer neuronal layer of the retina, which is a component of the central nervous system. The photoreceptor cells are well sheltered in the center of the eye, and are protected structurally by the sclera, nourished by the highly-vascularized uvea and safeguarded by the blood-retinal barrier of the retinal pigment epithelium.

The primary function of the photoreceptor cells is to convert light into a physio-chemical signal (transduction) and to transmit this signal to the other neurons (transmission). During the transduction and transmission processes, the metabolic activities of these neurons are changed dramatically. Even though the photoreceptor cells are securely protected in the interior of the eye, these cells are readily accessible to light because their primary function is light detection. Excessive light energy reaching the retina can cause damage to these neurons, either directly or indirectly, by overwhelming the metabolic systems of these cells.

A number of natural mechanisms protect the photoreceptor cells from light injury. For example, the ocular media, including the cornea, aqueous, lens, and vitreous, filter most of the light in the ultraviolet region. However, after cataract extraction or other surgical intervention, some of these protective barriers are removed or disturbed, whereby the photoreceptor cells are more susceptible to damage by radiant energy. The photoreceptor cells also possess other forms of protection from photic injury, for example, the presence of antioxidant compounds to counteract the free radical species generated by light. In addition, the human eye has an excessive number of photoreceptor cells such that only destruction of a significant number of photoreceptor cells adversely affects visual function.

A number of different classes of compounds have been reported to minimize retinal photic injury in various animal models: antioxidants, such as, ascorbate (Organsciak et al. 1985), dimethylthiourea (Organisciak et al. 1992; Lam et al. 1990), α-tocopherol (Kozaki et al. 1994), and β-carotene Rapp et al. 1995); calcium antagonists, such as, flunarizine, (Li et al. 1993; Edward et al. 1992); growth factors, such as, basic-fibroblast growth factor, brain derived nerve factor, ciliary neurotrophic factor, and interleukin-1-.beta. (LaVail et al. 1992); glucocorticoids, such as, methylprednisolone (Lam et al. 1993), dexamethasone (Fu et al. 1992); N-methyl-D-aspartate (NMDA) antagonists (U.S. Pat. No. 6,509,355) and iron chelators, such as, desferrioxamine (Li et al. 1991).

What is needed is a method of treatment of neurodegeneration resulting from photic injury that addresses the mechanism whereby photoreceptor cell death occurs.

### SUMMARY OF THE INVENTION

The present invention overcomes these and other drawbacks of the prior art by providing a method of preventing neurodegeneration resulting from photic injury by administering to a patient or a mammal a therapeutically effective amount of an inhibitor of tumor necrosis factor-converting enzyme (TACE). While it is believed that any compound that inhibits TACE will be useful in the methods of the invention, the preferred TACE inhibitor is IC-3.

### BRIEF DESCRIPTION OF THE DRAWINGS

The following drawings form part of the present specification and are included to further demonstrate certain aspects of the present invention. The invention may be better understood by reference to these drawings in combination with the detailed description of specific embodiments presented herein.

FIG. 1A. Increased expression and cleavage of p75NTR in light-exposed photoreceptor cells. 661 w cells were exposed to light for 0-3 h, labeled with anti-p75ICD antiserum (9992) at 1:200 dilution, and observed under phase or fluorescent microscopy. After 3 h of exposure, cells were rounded and much smaller in size suggestive of cell death. Increased staining for p75NTR [green] was observed with increased exposure and appeared to co-localize with DAPI [blue] in the nuclear areas after 2-3 h exposure.

FIG. 1B. Protein levels of p75NTR were determined by Western blot and densitometric analyses of 661 w lysates using 1:2000 dilutions of the anti-p75ICD (9992) or the anti-p75ECD (9651) antisera. Bands at ˜75 kDa were reactive with both antisera, while ˜50 kDa bands reacted only with the anti-p75ICD. β-tubulin was used to normalize protein loading.

FIG. 1C. Subcellular fractionation followed by Western blots were also done utilizing either antisera to determine nuclear distribution of the ˜50 kDa p75ICD. With longer light exposures, increased intensity of the ˜75 kDa bands was observed with either antiserum in both membrane and cytosolic fractions, but not in the nuclear extracts. The ˜50 kDa p75ICD-reactive bands were absent in the membrane fraction but appeared in the cytosolic fractions as early as 1 h of light exposure and in the nuclear fractions after 2-3 h light exposure. The purity of the fractions was verified by probing for β-actin (membrane), splicing factor (nuclear) and Akt (cytosolic).

FIG. 2A. Light exposure promotes increased expression of TACE. 661 w cells exposed to light for 0-3 h were immunostained for TACE using rabbit polyclonal anti-TACE antiserum at 1:200 dilution, and observed under phase contrast or fluorescence microscopy. TACE immunostaining [green] appeared to increase with light exposure in cultured cells, especially in small rounded cells exposed for 3 h.

FIG. 2B. Protein levels of TACE were determined by Western blot analysis of 661 w lysates using rabbit anti-TACE antiserum at 1:500 dilution and showed significant increase with longer light exposures. A single TACE-reactive band ˜70 kDa in size was observed in the untreated cells while multiple bands reactive for TACE ˜40-70 kDa were seen following light exposure. β-tubulin was used to normalize protein loading.

FIG. 3A. Coexpression of TACE and p75NTR promotes p75NTR cleavage. 661 w cells stably transfected with empty vector (lane 1), full length p75NTR (lane 2), full length TACE (lane 3), or TACE and p75NTR together (lane 4) were lysed and probed by Western blot using rabbit polyclonal anti-TACE antiserum at 1:500 dilution or anti-p75ICD antiserum (9992) at 1:2000 dilution. Increased levels and presence of multiple TACE-reactive bands ˜40-70 kDa were seen in TACE-stable transfectants [lanes 3 & 4].

FIG. 3B. p75NTR-transfected cells showed increased expression of 75 kDa p75NTR-reactive bands. A strong ˜50 kDa p75ICD-reactive band, that was not recognized by the anti-p75ECD (data not shown) was also observed only in lysates of cells co-transfected with TACE and p75NTR [lane 4].

FIG. 3C. A ˜50 kDa band reactive with the anti-p75ECD antiserum diluted 1:2000, but not with the anti-p75ICD diluted 1:2000 (data not shown) was identified in the 24 h-conditioned media of cells co-expressing both TACE and p75NTR [lane 4].

FIG. 3D. Subcellular fractionation followed by Western blot using anti-p75ICD antiserum at 1:2000 showed ˜75 kDa bands in the membrane and cytosolic fractions of p75NTR-overexpressing cells [lanes 2 & 4] and a ˜50 kDa band in the cytosolic fractions but not in the membrane nor nuclear fractions of cells co-transfected with p75NTR and TACE [lane 4]. The fractions were tested for purity using B-actin (membrane), splicing factor (nuclear) and Akt (cytosolic).

FIG. 4A. TAPI, a TACE inhibitor, blocks cleavage of p75NTR and inhibits light-induced cell death. 661 w cells exposed to light for 0-3 h in the presence or absence of 5 μM IC-3, were lysed and/or fractionated, then probed with anti-p75ICD antiserum (9992) diluted 1:2000 or anti-TACE antiserum diluted at 1:500. β-tubulin was used to normalize protein loading. Immunoblots of cell lysates showed increased expression of a ˜75 kDa reactive band in both IC-3 treated and untreated cultures. A ˜50 kDa p75ICD-reactive band observed to increase only in light-exposed cells was not seen following IC-3 treatment.

FIG. 4B. Immunoblots of subcellular fractions of light-damaged 661 w cells showed a similar increase in the intensity of the ˜75 kDa band in membrane and cytosolic fractions, but not in the nuclear fractions. The ˜50 kDa p75ICD-reactive band observed in the cytosolic fraction of untreated cultures was absent in all fractions of IC-3 treated cultures.

FIG. 4C. 661 w cells exposed to light for 0-3 h in the presence or absence of 5 mM IC-3, were lysed and probed with anti-TACE antiserum diluted at 1:500. α-tubulin was used to normalize protein loading. Multiple TACE-reactive bands ˜40-70 kDa in size increased with light exposure and appeared similar in both IC-3-treated and untreated cultures.

FIG. 4D. To determine the effect of IC-3 on light-induced cell death, 661 w cells were exposed to light for 0-3 h in the presence of 0-10 μM IC-3 and assayed for cell survival by MTS assay. IC-3, at concentrations ranging from 1-10 μM, protected 661 w cells from cell death due to intense light exposure for 2-3 h. In the presence of 5 μM IC-3, the amounts of cell death after 2-3 h light exposure were not significantly different from control cultures or cultures exposed to light for 1 h.

FIG. 4E. To establish that light-induced cell death was apoptotic, 661 w cells were exposed to light for 0-3 h in the absence of 5 μM IC-3, stained with 40 μg/ml of propidium iodide (PI), and DNA content was analysed by flow cytometry. Experiments were done in triplicate at least three times and analysed using ANOVA. The subG1 population, reflective of apoptotic cells, increased from 0.89% in control 661 w cultures to 19.01% and 27.76% in cells exposed to light for 2 h and 3 h, respectively.

FIG. 4F. To determine whether the TACE inhibitor IC-3 suppresses light-induced cell death, 661 w cells were exposed to light for 0-3 h in the presence of 5 μM IC-3, stained with 40 μg/ml of propidium iodide (PI), and DNA content was analysed by flow cytometry. Experiments were done in triplicate at least three times and analysed using ANOVA. In the presence of IC-3, the subG1 peak decreased to about 2.98% in cells exposed to light for 3 h as compared with 27.76% in the absence of IC-3, consistent with the protective effect of IC-3.

FIG. 5A. and FIG. 5B Expression of p75NTR (FIG. 5A) and TACE (FIG. 5B) in light-damaged rat retinas. To determine the effects of light exposure on the expression of p75NTR and TACE in vivo, free-moving normal Sprague-Dawley rats were exposed to blue light (220 fc) for 6 h and their retinas were processed for immunostaining for p75NTR and TACE following 24 h or 5 days of recovery in the dark. In normal rat retinas (control), immunolabeling for p75NTR and TACE was very low, with p75NTR (FIG. 5A) mostly distributed in the inner retina around ganglion cells, while TACE (FIG. 5B) localized to the retinal pigment epithelium (RPE). Increased staining for both p75NTR and TACE in the retina was observed with light exposure and 24 h recovery. p75NTR and TACE immunoreactivity was observed in radial Müller cell processes and photoreceptor outer segments (OS). In the RPE, p75NTR staining increased, while the levels of TACE decreased. With light exposure and longer recovery of 5 days, the photoreceptor cell layer appeared thinner and the p75NTR and TACE were much more intense especially in the inner retina. In the RPE, p75NTR staining was stronger than in other areas, while TACE was almost gone.

### DETAILED DESCRIPTION PREFERRED EMBODIMENTS

Photic retinopathy results from excessive excitation of the retinal pigment epithelium and neuroretina by absorption of visible or near ultraviolet radiation. Lesion severity is dependent upon wavelength, irradiance, exposure duration, species, ocular pigmentation, and age. Damage may result from peroxidation of cellular membranes, inactivation of mitochondrial enzymes such as cytochrome oxidase, or increased intracellular calcium. Cellular damage resulting from photo-oxidative stress leads to cell death by apoptosis, (Shahinfar et al. 1991; Abler et al. 1994). Oxidative stress induced apoptosis has been implicated as a cause of many ocular pathologies, including, iatrogenic retinopathy, macular degeneration, retinitis pigmentosa and other forms of heredodegenerative disease, ischemic retinopathy, retinal tears, retinal detachment, glaucoma and retinal neovascularization (Chang et al. 1995; Portera-Cailliau et al. 1994; Buchi 1992; Quigley et al. 1995). Photic induced retinal damage has been observed in mice (Zigman et al. 1975), rats (Noell et al. 1966; Kuwabara and Gorn 1968; LaVail 1976), rabbit (Lawwill 1973), squirrel (Collier and Zigman 1989; Collier et al. 1989), non-human primates (Tso 1973; Ham et al. 1980; Sperling et al. 1980; Sykes et al. 1981; Lawwill 1982), and man (Marshall et al. 1975; Green and Robertson 1991). In man, chronic exposure to environmental radiation has also been implicated as a risk factor for age-related macular degeneration (Young 1988; Taylor et al. 1992; Cruickshanks et al. 1993). The eye is exposed to high-energy laser radiation during the performance of retinal photocoagulation therapy (grid, focal and panretinal) or during photodynamic therapy. This type of therapy is often employed during treatment of choroidal neovascularization, proliferative stages of diabetic retinopathy, retinopathy of prematurity, or to repair retinal holes or detachments. Associated with this laser therapy is tissue destruction leading to vision deterioration. The Macular Photocoagulation Study found that 20% of the eyes treated for subfoveal macular choroidal neovascularizations (CNV) and 18% of the eyes treated for juxtafoveal CNV suffered severe visual loss of six or more lines as a direct result of laser treatment. It is believed that this vision loss results directly from the expansion of the laser-induced lesion to surrounding normal neurosensory retina and RPE. Singlet oxygen and other reactive oxygen species as well as cytokines are generated in the area of the laser burn and thought to migrate laterally to cause collateral retinal damage. Retinal morphology changes in this area are similar to changes in our photo-oxidative retinopathy paradigm.

p75NTR is a member of tumor necrosis factor receptor (TNFR) superfamily, that includes CD27, CD30, CD40, OX40, Fas/CD95, TNFR-1 and TNFR-2 (Baker and Reddy 1998; Miller and Kaplan 1998), and can induce cell death both in vitro and in vivo (Barrett and Barlett 1994; Casaccia-Bonnefil et al. 1996; Frade and Barde 1999; Majdan et al. 1997; Frade et al. 1996; Coulson et al. 2000). p75NTR contains a cysteine-rich extracellular domain (p75ECD) and a death domain-containing intracellular portion (p75ICD) (Barker 1998). The death domain is a type II domain (Roux and Barker 2002) and does not aggregate or self-associate like the Fas/CD95 death domain nor bind other death domain containing proteins (Liepinsh et al. 1997; Wang et al. 2001) to signal for cell death. Instead, studies show that overexpression of p75ICD induces neuronal cell death within the central and peripheral nervous systems (Majdan et al. 1997; Coulson et al. 2000) suggesting that cleavage and release of the p75ICD might be involved in p75NTR-mediated cell death. The full length p75NTR has been shown to be cleaved by a constitutively active membrane-bound metalloprotease to generate a soluble p75ECD and the membrane-bound receptor fragment containing the p75ICD, which is highly conserved with complete homology among species such as chick, rat and human (Heuer et al. 1990a; Heurer et al. 1990b; Zupan et al. 1989; Barker et al. 1991; Zupan and Johnson 1991; DiStefano et al. 1993). To date, the exact nature of the enzyme(s) responsible for cleavage of p75NTR has not been determined.

Tumor necrosis factor-converting enzyme (TACE) is a member of A disintegrin and metalloprotease (ADAM) family (Schlondorff and Blobel 1999) of transmembrane glycoproteins that contain both a disintegrin and a metalloprotease domain (Black and White 1998). These proteins have been implicated in various cellular processes such as matrix degradation, cell migration, cell-cell interaction and ectodomain shedding of cytokines and growth factors from membrane-bound precursors (Schlondorff and Blobel 1999). TACE, which is expressed constitutively in many tissues (Black et al. 1997), is a major proteolytic enzyme for tumor necrosis factor-α (Black et al. 1997; Moss et al. 1997), but also promotes cleavage of a diverse group of transmembrane proteins including transforming growth factor-α (Peschon et al. 1998), L-selectin (Zhang et al. 2000), p75 tumor necrosis factor receptor (Peschon et al. 1998), growth hormone receptor (Zhang et al. 2000), amyloid precursor protein (APP) (Buxbaum et al. 1998) and prions (Vincent et al. 2001) resulting in shedding of the soluble extracellular domain. Moreover, TACE cleavage of Notch, amyloid precursor protein (APP), and ErbB-4 receptor initiates the release and nuclear translocation of their respective intracellular domains in a process called regulated intramembrane proteolysis (Heldin and Ericsson 2001). Due to its high homology to TNFR, p75NTR appears to be a suitable substrate for TACE-induced cleavage that may result in both shedding of a soluble p75ECD and generation of a p75ICD that has been implicated in apoptosis.

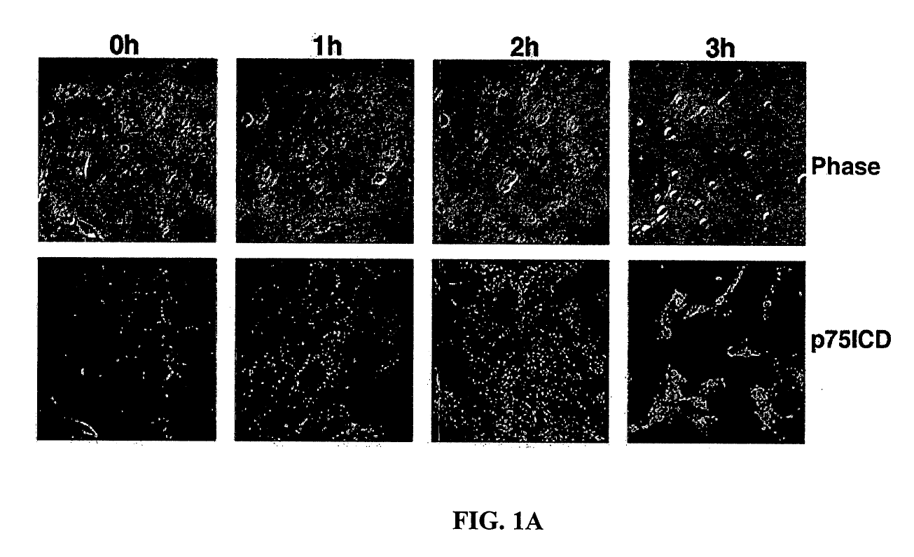

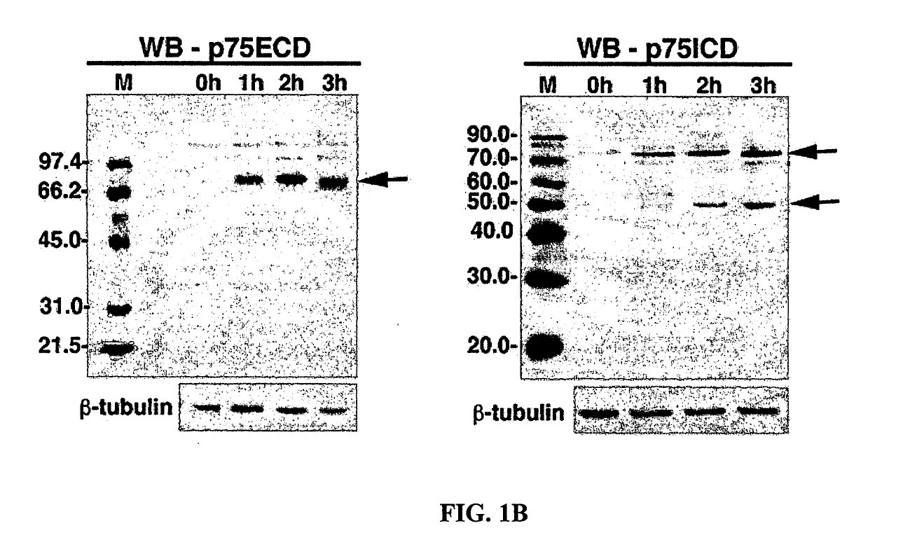

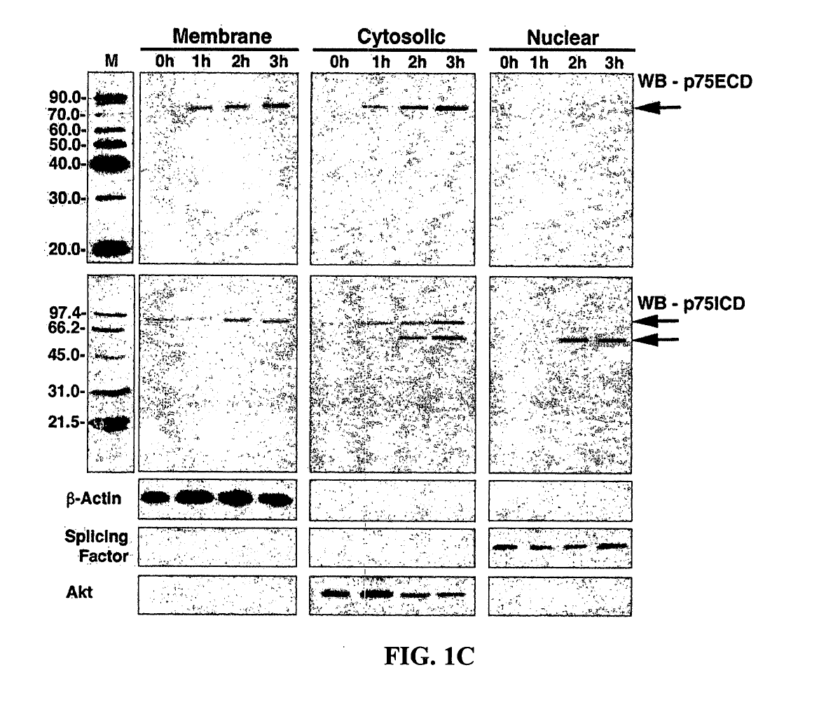

The present inventors investigated the effects of intense light exposure on the expression of p75NTR and TACE, as well as the ability of TACE to promote cleavage of p75NTR. A well characterized photoreceptor cell line, 661 w cells, isolated from retinal tumors of transgenic mice, has been shown previously to undergo photo-oxidative stress-induced apoptosis following light exposure (Al-Ubaidi et al. 1992; Krishnamoorthy et al. 1999). Exposure to white light at 1400 foot candles (fc) for at least 2 h results in apoptosis, although significant cell death is observed only after 3 h of exposure (Al-Ubaidi et al. 1992). 661 w cells were exposed to light for 0-3 h and tested for expression of p75NTR protein using immunocytochemistry. Staining for p75NTR was distributed primarily in the cytoplasm of untreated cultures. With prolonged exposure, increased staining was observed in the cytoplasm as well as in the nucleus as shown by the colocalization of p75NTR with DAPI, a nuclear stain, especially in cultures exposed for 2-3 h [FIG. 1A]. Images of p75NTR and DAPI colocalization were also sectioned serially at 0.5 μm thickness for Z-scan to verify nuclear distribution of both p75NTR and DAPI (Krishnamoorthy et al. 1999). The increased p75NTR protein levels were verified by Western blot and densitometric analyses using antiserum raised against p75ICD (9992) or against p75ECD (9651). Western blots showed increased intensity of 75 kDa bands reactive with either antiserum following light exposure, with a significant increase in the p75NTR:β-tubulin ratios in densitometric analyses (Roque et al. 1999). Western blots also exhibited increased levels of 50 kDa bands in blots probed for the p75ICD [FIG. 1B], suggesting that the ˜50 kDa band is a p75NTR fragment containing the intracellular but not the extracellular domain. Whether the ˜50 kDa bands result from molecular splicing or from post-translational processing of the full length p75NTR remains to be established.

To confirm the nuclear localization of p75NTR, subcellular fractionation was performed on light-damaged 661 w cells followed by Western blot analyses. Antibodies against major proteins in each fraction were used to assess the purity of each fraction as well as to normalize loading of samples. With longer light exposures, blots using either antiserum showed increased intensity of the ˜75 kDa bands in both membrane and cytosolic fractions, but not in the nuclear extracts. [FIG. 1C]. The ˜50 kDa p75ICD-reactive bands were absent in the membrane fraction but appeared in the cytosolic fractions as early as 1 h of light exposure and in the nuclear fractions after 2-3 h light exposure, suggestive of nuclear translocation of the ˜50 kDa p75ICD.

The full length p75NTR (p75FL) also has been shown to be cleaved by a constitutively active membrane-bound metalloprotease to generate a soluble p75ECD and a membrane-bound receptor fragment containing the transmembrane domain and the p75ICD (Zupan et al. 1989; Barker et al. 1991; Zupan and Johnson 1991; DiStefano et al. 1993).

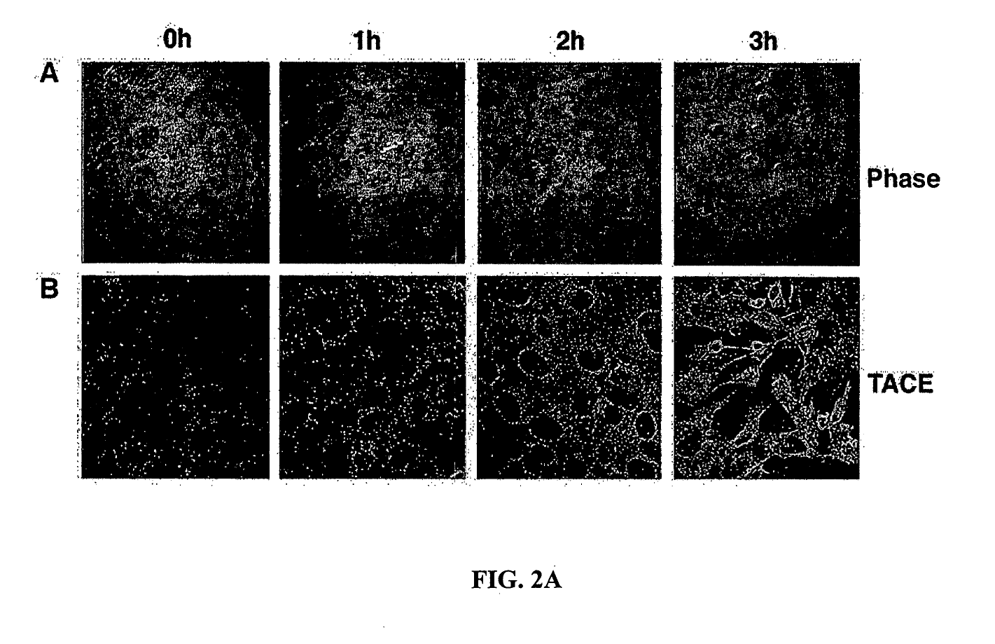

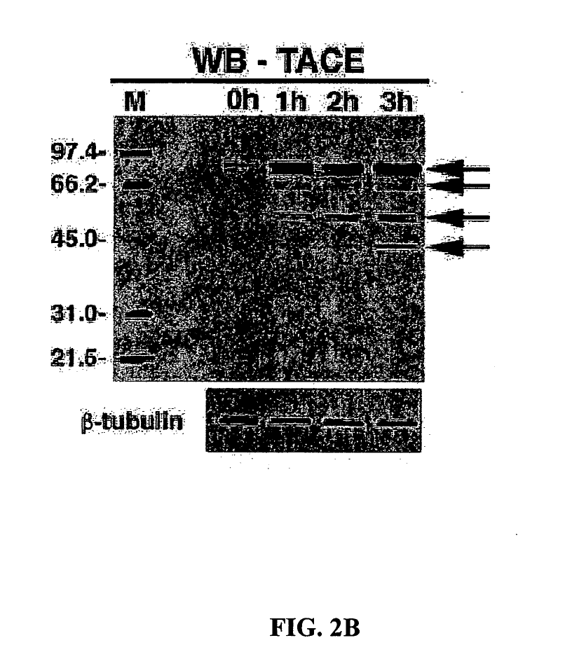

While the identity of the enzyme(s) responsible for cleavage of p75NTR has not been determined, the high homology of p75NTR to TNFR suggests that p75NTR might be a suitable substrate for TACE-induced proteolysis. The expression of TACE was investigated in 661 w cells following light exposure for 0-3 h. TACE immunostaining appeared to increase with light exposure in cultured cells [FIG. 2A] and in immunoblots [FIG. 2B]. Western blot and densitometric analyses of the TACE/β-tubulin ratio showed significant increase with longer light exposures (Black et al. 1997). A single TACE-reactive band ˜70 kDa in size was observed in the untreated cells while multiple bands reactive for TACE ˜40-70 kDa were seen following light exposure. The presence of multiple TACE bands in light exposed cells suggests either increased expression of novel molecular forms of TACE or proteolytic processing of TACE itself. In other cell types, the mature TACE migrates as a ˜100 kDa 100 kDa protein under reducing conditions (Schlondorff et al. 2000).

To establish whether the ˜50 kDa p75ICD reactive-band is a novel molecular form of p75NTR or a proteolytic product of TACE-mediated cleavage of the full length p75NTR, 661 w cells were stably transfected with plasmids containing the cDNAs for the full length p75NTR (p75FL), TACE, or both, and processed for Western blot for p75NTR and TACE.

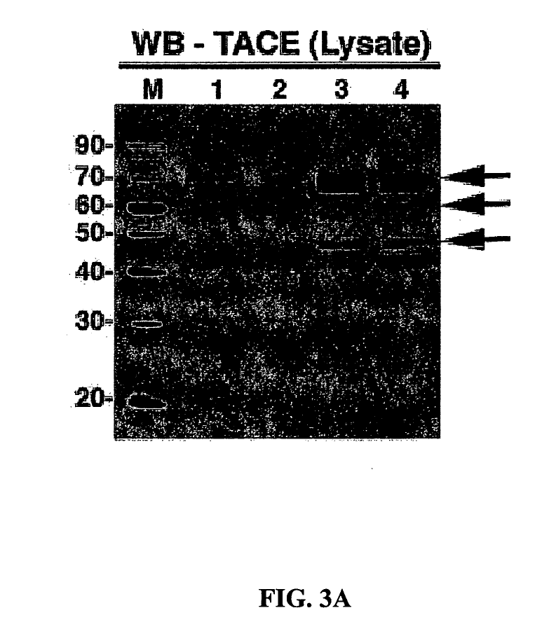

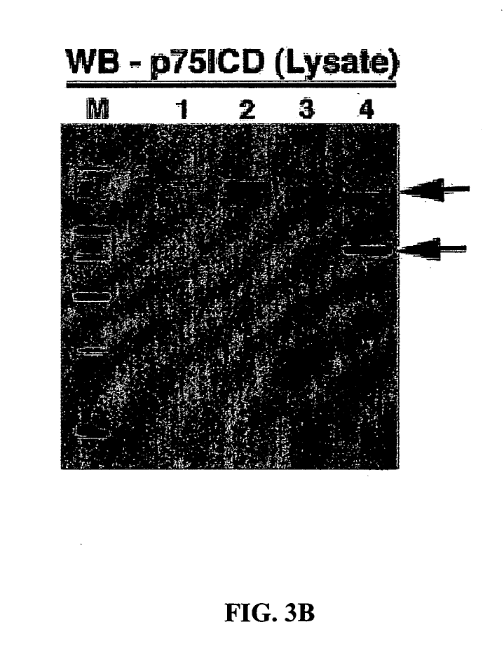

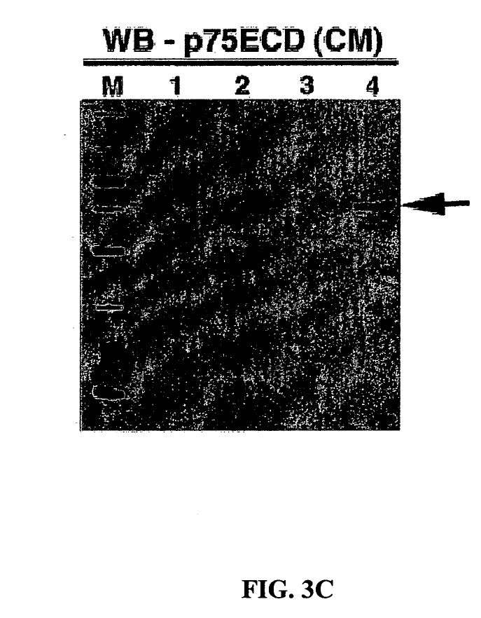

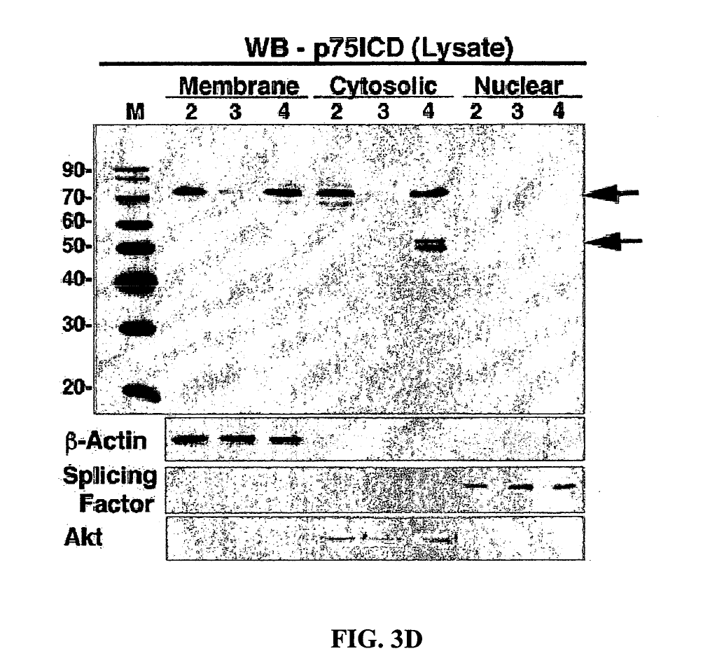

The expression of TACE [FIG. 3A] and the ˜75 kDa p75NTR-band [FIG. 3B] increased following transfection with the appropriate plasmids [FIG. 3A, lanes 3 & 4; FIG. 3B, lanes 2 & 4]. TACE-stable transfectants exhibited multiple TACE-reactive bands similar to those in light-damaged cultures. A strong ˜50 kDa p75ICD-reactive band [FIG. 3B, lane 4], that was not recognized by the anti-p75ECD (data not shown) was also observed only in lysates of cells transfected with both TACE and p75NTR. A 50 kDa p75ECD-reactive band [FIG. 3C, lane 4], that was not recognized by the anti-p75ICD (data not shown) was also observed only in the 24 h-conditioned media of 661 w cells co-expressing TACE and p75NTR. Soluble p75NTR ˜50 kDa and ˜45 kDa in sizes released in the spent media of cultured rat Schwann cells and A875 human melanoma cells, respectively, have been previously reported (Zupa and Johnson 1991; DiStefano et al. 1993). Protein purification of soluble p75NTR from human infant urine and amniotic fluid yielded 3 species with molecular masses of 45 kDa, 40 kDa, and 35 kDa (Zupan et al. 1989). These findings support our hypothesis that upregulation of TACE levels promoted ectodomain shedding of the p75ECD. Moreover, the appearance of the ˜50 kDa p75ICD-reactive band only in cells transfected with the full length p75NTR and TACE proves that the ˜50 kDa band is a proteolytic product of TACE cleavage, and not a spliced variant of p75NTR.

Subcellular fractionations followed by Western blot using anti-p75ICD antiserum showed ˜75 kDa bands in the membrane and cytosolic fractions of p75NTR-overexpressing cells and a ˜50 kDa band in the cytosolic fractions but not in the membrane nor nuclear fractions of cells co-transfected with p75NTR and TACE [FIG. 3D]. Thus, while TACE promoted the cleavage of the full length p75NTR and release of the p75ICD into the cytosol, TACE by itself did not promote nuclear translocation of p75ICD. This is consistent with the absence of a nuclear localization signal in p75NTR. The presence of the ˜50 kDa p75ICD in the nucleus of light-damaged cells [FIG. 1C] might be facilitated by increased expression of other proteins, i.e. adaptor proteins or chaperones, necessary to promote nuclear translocation of the p75ICD. The presence of 75 kDa band in the cytosolic fraction of p75NTR-tranfected cells, similar to that in light-damaged cells [FIG. IC], could be due to the release of endosomal p75NTR during cell disruption and subcellular fractionation or from endosomal contamination of the preparations. Internalization of p75NTR via clathrin-coated pits into early endosomes and vesicles has been reported in glial cells and PC12 cells (Kahle and Hertel 1992; Bronfinan et al. 2003).

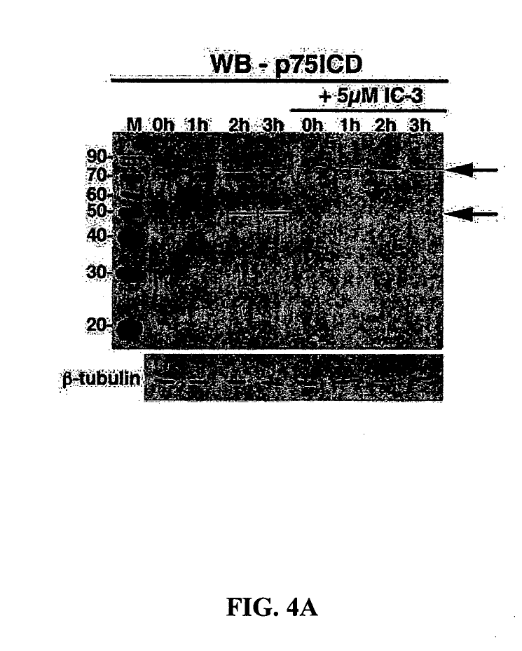

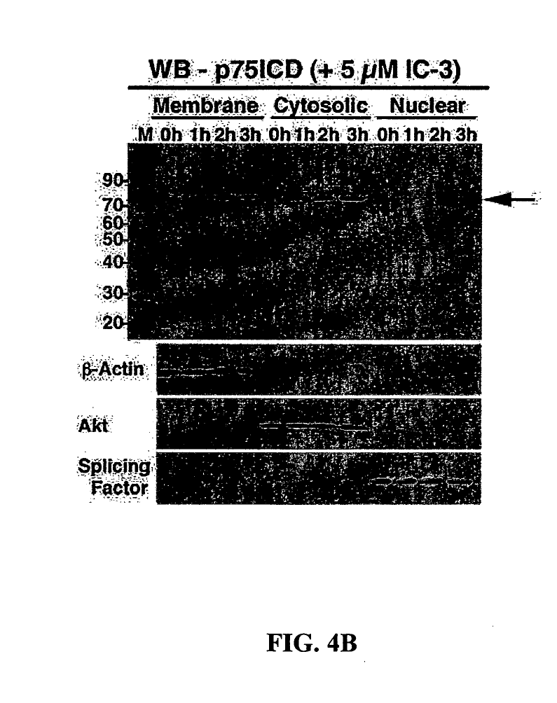

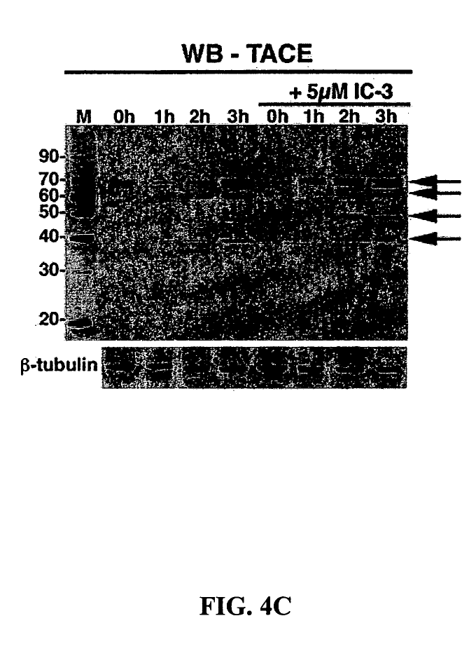

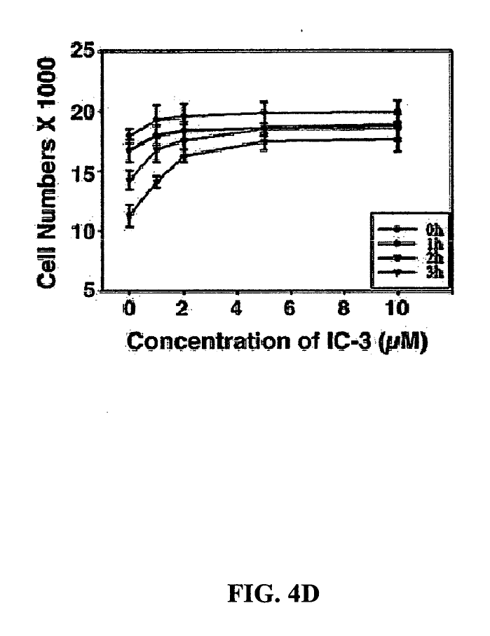

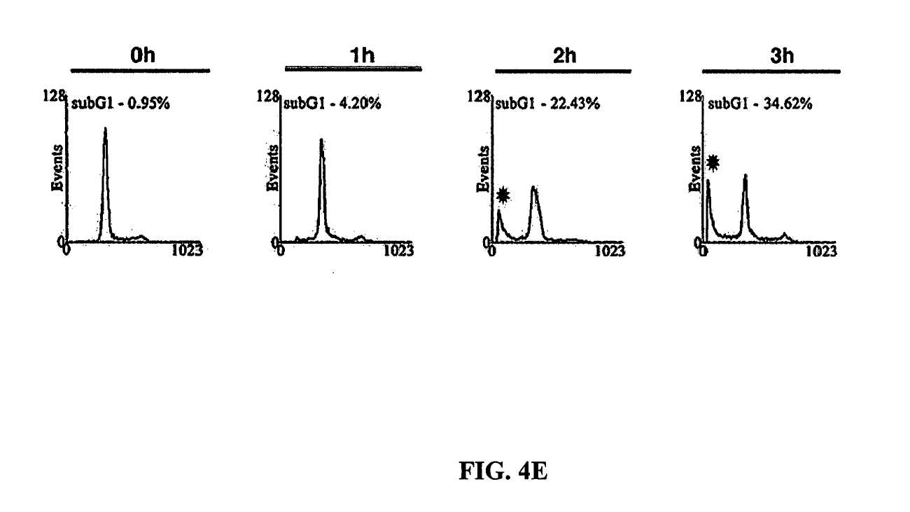

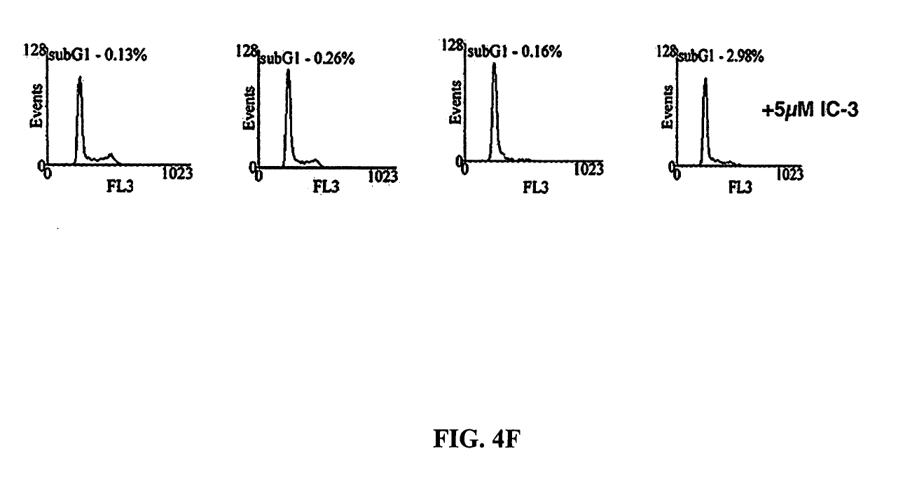

The effects of light-induced TACE-cleavage of p75NTR were further tested by exposing 661 w cells to light for 0-3 h in the presence of 5 μM Immunex Compound-3 (IC-3), a TACE inhibitor, and probed for p75NTR or TACE by Western blot analysis. Immunoblots of light-exposed cells treated with IC-3 showed increased expression of the 75 kDa band as in untreated cultures [FIG. 4A], however, the ˜50 kDa p75ICD-reactive band in light-exposed cells was absent following IC-3 treatment. Immunoblots of subcellular fractions showed a similar increase in the intensity of the ˜75 kDa band in membrane and cytosolic fractions, but not in the nuclear fractions, as well as the absence of the ˜50 kDa p75ICD-reactive band in all fractions of IC-3 treated cultures [FIG. 4B]. Multiple TACE-reactive bands increased with light exposure and appeared similar in both IC-3-treated and untreated cultures [FIG. 4B].

These suggest that IC-3 did not inhibit the stimulatory effects of light exposure on the expression of TACE and the full length p75NTR, but suppressed the TACE-mediated cleavage of p75NTR. While TACE itself may not promote nuclear translocation of the p75ICD, cleavage of p75NTR by TACE appeared to be necessary for its nuclear localization. The presence of multiple TACE bands in the TACE-overexpressing cells further suggest that the increased TACE expression during light exposure might stimulate cleavage of TACE by TACE itself or other proteases yet to be identified, and not from alternative splicing. Since IC-3 inhibited proteolytic cleavage of p75NTR, thereby preventing release and nuclear translocation of the p75ICD, IC-3 was used to determine whether nuclear translocation of p75ICD was necessary for light-induced apoptosis. Light-exposed cells were treated with IC-3 and tested for survival or apoptosis using MTS assay or FACS analysis, respectively. IC-3, at concentrations ranging from 1-10 μM, protected 661 w cells from cell death due to intense light exposure for 2-3 h [FIG. 4D]. In the presence of 5 μM IC-3, the amount of cell death after 2-3 h light exposure was not significantly different from control cultures or cultures exposed to light for 1 h. In FACS analyses, the subG1 population, reflective of apoptotic cells, increased from 0.89% in control cultures to 19.01% and 27.76% in cells exposed to light for 2 h and 3 h, respectively [FIG. 4E]. In the presence of IC-3, the subG1 peak decreased to about 2.98% in cells exposed to light for 3 h [FIG. 4F]. These data suggest that IC-3 inhibited light-induced apoptotic cell death in cultured photoreceptor cells.

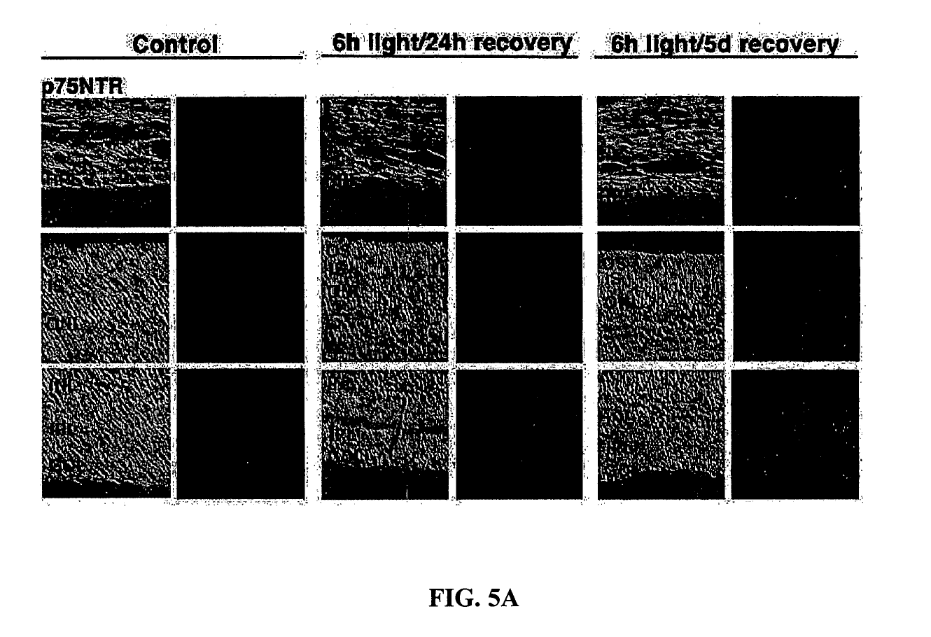

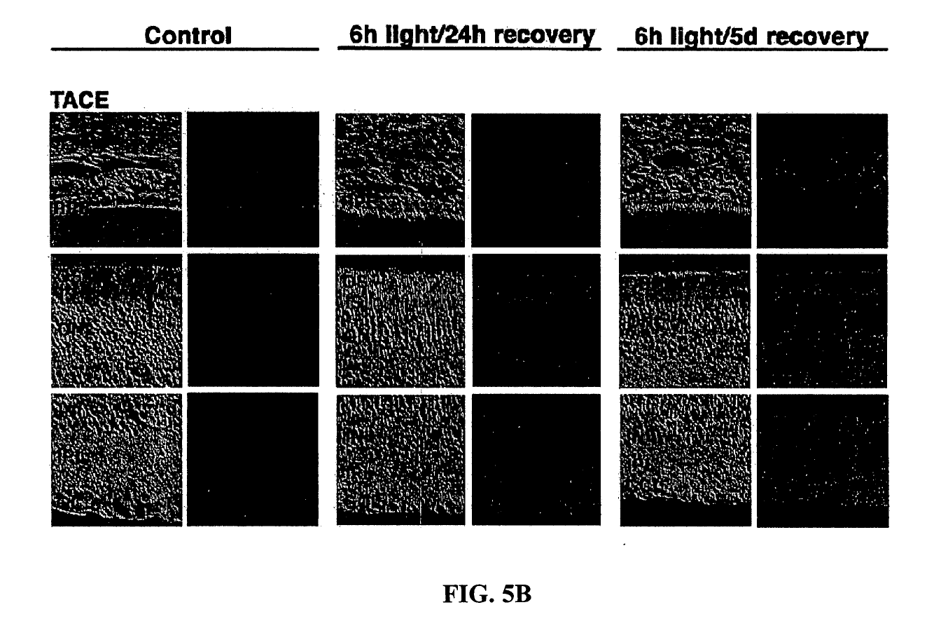

To establish the effects of light exposure on the expression of p75NTR and TACE in animal retinas, free-moving normal Sprague-Dawley rats were exposed to light for 6 h and their retinas were processed for immunostaining for p75NTR and TACE following 24 h or 5 days of recovery in the dark [FIG. 5]. In normal rat retinas (control), immunoreactivity for p75NTR and TACE was negligible, with p75NTR mostly distributed in the inner retina around ganglion cells, while TACE localized to the retinal pigment epithelium (RPE). Increased staining for both p75NTR and TACE in the retina was observed with light exposure and 24 h recovery. Although the strongest labeling was observed in the inner retina especially in radial processes, probably of Müller cells, p75NTR and TACE immunoreactivity was also observed in the outer segments (OS) of photoreceptor cells. In the RPE, while p75NTR staining increased in intensity, decreased levels of TACE were observed. With light exposure and longer recovery of 5 days, the photoreceptor cell layer appeared thinner and the p75NTR and TACE were much more intense especially in the inner retina. In the RPE, p75NTR staining was stronger than in other areas, while TACE was almost absent. The increased expression of TACE and p75NTR in photoreceptor cells of animal retinas with photic injury suggest that TACE and p75NTR might be involved in light-induced photoreceptor cell death. The present inventors have discovered that light exposure stimulates increased expression of p75NTR and TACE in cultured cells. While p75NTR expression has been shown to be upregulated in various genetic and environmental conditions (Sheedlo et al. 2002; Harada et al. 2000; Kokaia et al. 1998; Roux et al. 1999; Oh et al. 2000), the present inventors are the first to establish a direct relationship between light-induced apoptosis and proteolytic cleavage and nuclear translocation of p75NTR. While overexpression of the p75ICD (Majdan et al. 1997; Coulson et al. 2000) has been shown previously to promote neuronal apoptosis, the present inventors describe for the first time a nuclear signaling mechanism utilized by p75ICD in promoting apoptosis.

The present inventors are also the first to report light-mediated upregulation of TACE expression. TACE is constitutively expressed in various tissues and the proteolytic activity and degradation of TACE are both induced by various molecules such as phorbol esters via protein kinase C (Heldin and Ericsson 2001; Black 2002), however, the mechanisms of increased TACE expression remain unknown. Moreover, while a constitutively expressed membrane-bound metalloprotease is believed to be responsible for ectodomain shedding of p75NTR (DiStefano et al. 1993), the present inventors are the first to suggest p75NTR is a substrate for TACE. The present inventors have shown that during light damage, TACE promotes cleavage of full length p75NTR resulting in shedding of the ˜50 kDa p75ECD and release and nuclear translocation of the p75ICD. TACE has been shown to initiate regulated intramembrane proteolysis (RIP) of several molecules such as Notch, APP, and ErbB-4 receptor by a cleavage within the extracellular domain (Heldin and Ericsson 2001). This is followed by cleavage of the membrane-bound portion in the transmembrane domain by a γ-secretase, usually presenilin, to release the cytoplasmic domain that translocates to the nucleus. It is possible that other proteases, including γ-secretases, are involved in the proteolytic cleavage and nuclear translocation of p75NTR, however, the short duration of exposure of blots in the photo-imaging system utilized in our laboratory allows only the major bands to develop. Exposing the blots for longer durations often resulted in numerous bands, which might have been additional cleavage products, however, their specificity was difficult to ascertain. Cleavage products lacking the epitopes recognized by the antisera could also have led to missing other products. Additional studies will be needed to determine whether p75NTR undergoes additional downstream proteolytic cleavage, such as by γ-secretases, during light exposure. However, the similarity in size of the p75ICD species in the nucleus of light-exposed cells and the size of the p75ICD in TACE and p75NTR co-transfected cells suggest that TACE cleavage is enough to initiate the signaling pathway promoting nuclear translocation of p75ICD.

p75NTR was found in the nuclei of light-exposed 661 w cells, but in the presence of IC-3, nuclear translocation of p75NTR was inhibited, suggesting that prior processing of p75NTR by TACE is required for its nuclear translocation. In cells overexpressing TACE and p75NTR, the products of TACE cleavage were present but the p75ICD did not translocate to the nucleus consistent with the absence of a nuclear localization signal in p75NTR. Preliminary studies show that NRAGE, a p75NTR-adaptor protein, and p53, a transcription factor, are necessary for the nuclear shuttling of p75NTR in light-induced apoptosis (data not shown). In general, for degenerative diseases, the compounds of this invention are administered orally with daily dosage of these compounds ranging between 0.01 and 500 milligrams. The preferred total daily dose ranges between 1 and 100 milligrams. Non-oral administration, such as, intravitreal, topical ocular, transdermnal patch, parenteral, intraocular, subconjunctival, or retrobulbar injection, iontophoresis or slow release biodegradable polymers or liposomes may require an adjustment of the total daily dose necessary to provide a therapeutically effective amount of the compound. The compounds can also be delivered in ocular irrigating solutions used during surgery (see U.S. Pat. No. 5,604,244 for irrigating solution formulations).

Concentrations should range from 0.001 mM to 10 mM, preferably 0.01 mm to 5 mM. The compounds can be incorporated into various types of ophthalmic formulations for topical delivery to the eye. They may be combined with ophthalmologically acceptable preservatives, surfactants, viscosity enhancers, penetration enhancers, buffers, sodium chloride, and water to form aqueous, sterile ophthalmic suspensions or solutions.

Ophthalmic solution formulations may be prepared by dissolving the compound in a physiologically acceptable isotonic aqueous buffer. Further, the ophthalmic solution may include an ophthalmologically acceptable surfactant to assist in dissolving the compound. The ophthalmic solutions may contain a thickener, such as, hydroxymethylcellulose, hydroxyethylcellulose, hydroxypropylmethylcellulose, methylcellulose, polyvinyl-pyrrolidone, or the like, to improve the retention of the formulation in the conjunctival sac.

In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-940, or the like, according to the published formulations for analogous ophthalmic preparations; preservatives and tonicity agents can be incorporated. If dosed topically, the compounds are preferably formulated as topical ophthalmic suspensions or solutions, with a pH of about 4 to 8. The compounds will normally be contained in these formulations in an amount 0.001% to 5% by weight, but preferably in an amount of 0.01% to 2% by weight. Thus, for topical presentation, 1 to 2 drops of these formulations would be delivered to the surface of the eye 1 to 4 times per day according to the routine discretion of a skilled clinician.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Cell Culture

A photoreceptor cell line (661 w cells) obtained from transgenic mouse retinas expressing SV40 T-antigen (Baker and Reddy 1998) was found to maintain photoreceptor characteristics such as specific cell markers and photo-oxidative pathways (Baker and Reddy 1998; Miller and Kaplan 1998; Barrett and Bartlett 1994). 661 w cells were grown to 80% confluency in growth medium (Dulbecco's modified Eagles medium, 2 mM L-Glutamine, 100 Units/ml Penicillin, 100 μg/ml Streptomycin, 15 mM HEPES buffer, and 10% fetal bovine serum) then transferred to serum-free medium for 24 h and exposed to light at 1400 foot-candles (fc) for 0-5 h. Cells were returned to the incubator for additional 24 h following light exposure. Cells maintained under similar conditions without light exposure were used as control. The effects of TACE on 661 w cells were verified using 0-50 μM IC-3 (Immunex compound 3; Immunex Corporation, Seattle, Wash.), a TACE inhibitor.

Transfection Experiments

Plasmids containing cDNA of the full length murine TACE (pcDNA3muTACEFL) (Casaccia-Bonnefil 1996) and/or the full length human p75NTR (pcDNA3hup75FL) [a gift from Dr. Phil Barker, McGill University, Montreal, Canada], or the empty vector alone were used for transfections. Cells were grown to 75% confluency and washed once with serum-free medium. Cells were stably transfected with 2 _g of plasmid using a mixture of LipofectAMINE plus (Life Technologies, Inc. Carlsbad, Calif.) and Nupherin-neuron (Biomol Research Laboratories Inc., Plymouth meeting, PA) in serum-free medium. Culture medium was replaced with growth medium after 4 h of incubation at 37° C. Transfected cells were selected using 800 μg/ml G418 and maintained in 200 g/ml G418.

Immunocytochemistry

Cells were fixed in 2% paraformaldehyde for 20 min and permeabilized with 0.1% Triton X-100 in phosphate buffered saline (PBS; 10 mM phosphate [pH 7.4], 150 mM NaCl) containing 0.05M glycine for 15 min. Cells were incubated in 5% BSA and 5% normal goat serum for 1 h at room temperature (RT) followed by 1:2000 dilution rabbit antiserum against the murine TACE cytoplasmic domain [AL45] (Frade and Barde 19995); or rabbit antisera against p75ECD [9651] or p75ICD [9992] (Majdan et al. 1997) at 4° C. overnight. After several rinses, cells were incubated with 10 μg/ml anti-rabbit IgG Alexa 488 or 594 (Molecular Probes Inc, Eugene, Oreg.) for 1 h, then counterstained with 300 nM DAPI (Molecular Probes Inc.) for 10 min. After PBS rinses, cover slips were mounted in aqueous mounting media and images of representative fields were captured in Nikon microphot FXA microscope with epifluorescent attachment (Nikon Corp., Tokyo, Japan). Images were further analyzed by serial sectioning and deconvolution of a stack of 20 images (0.5 μM thickness) under similar brightness and contrast settings.

Live Cell/Dead Cell Cytotoxicity Assay

The effect of light exposure on photoreceptor cell death/cell survival was determined using fluorescent probes calcein AM and ethidium homodimer (Molecular Probes Inc.) as described (Frade et al. 1996). 661 w cells plated at 10,000 cells/well on a 24-well plates were treated under various conditions then incubated with 2 μM calcein AM and 4 μM ethidium homodimer at 37° C. for 45 min and viewed under Olympus inverted microscope with epifluorescent attachment (Olympus Optical Co Ltd, Tokyo, Japan).

To measure the amounts of cell death from light exposure, 661 w cells plated at 5,000 cells/well on 96-well plates were exposed to light for 0-3 h in the presence of 0-50 μM IC-3. Following light exposure, cells were further incubated with the IC-3 for 24 h at 37° C. The number of surviving cells was determined using the Cell titer 96 assay (Promega Corp, Madison, Wis.) as described (Barrett and Bartlett 1994). Briefly, cells were incubated in 333 μg/ml of MTS and 25 μM phenazine methosulfate and the absorbance measured at 490 nm. at 15 min intervals for 1 h. A standard curve was prepared from cells plated at 0-50,000 cells/well. Experiments were done at least three times in triplicate. Values were expressed in cell counts and subjected to statistical analyses using ANOVA.

Flow Cytometry (FACS)

DNA content was determined by flow cytometry using propidium iodide staining. Following light exposure, 661 w cells were harvested by trypsinization, resuspended in 1.0 ml of Dulbecco's phosphate-buffered saline (DPBS) (10 mM phosphate [pH 7.4], 150 mM NaCl), and fixed by addition of 2.5 ml ice-cold 100% ethanol while vortexing. Cells were stored in ethanol overnight at −20° C. prior to staining. Cells were pelleted at 1500 rpm for 10 min and resuspended in PBS containing 40 μg/ml propidium iodide and 100/μg/ml RNase A. Cells were incubated at 37° C. for 30 min prior to flow cytometric analysis using a Coulter EPICS XL/XL-MCL flow cytometer (Beckman Coulter Inc., Fullerton, Calif.). DNA content was determined from histograms using WinMDI (Windows Multiple Documentation Interface, The Scripps Research Institute, La Jolla, Calif.) by gating on an area versus width in dot plot to exclude cell debris and cell aggregates. The percentage of degraded DNA was determined by the number of cells with subdiploid DNA divided by the total number of cells examined under each experimental condition.

Propidium Iodide—Hoechst 33342 Staining

661 w cells were fixed with 2% paraformaldehyde in 0.1M sodium phosphate and stained by incubating in PBS containing 500 nM Hoechst 33342 and 500 nM propidium iodide at 37° C. for 10 min in dark. Stained cells were viewed and the images were photographed under the Nikon microphot FXA microscope (Nikon Corp., Tokyo, Japan) with appropriate filters to visualize blue fluorescence (Hoechst 33342) and red fluorescence (Propidium iodide).

Subcellular Fractionation

Cells from 100 mm² dishes were washed with ice-cold DPBS and scraped into 2 ml of DPBS containing protease inhibitors (1 mg/ml of aprotinin, 1 mM phenymethylsulfonylfluoride and 1 mM sodium orthovanadate). Cells were disrupted using Dounce homogenizer, and the lysate was centrifuged at 100,000×g for 1 h at 4° C. The supernatant containing the cytosolic fraction was separated from the pellet and centrifuged using sucrose gradient at 300,000×g for 30 min. The supernatant containing the membrane fraction was separated from the pellet containing the nuclear fraction. The various fractions were analysed for p75NTR expression by Western blot using the rabbit antiserum against p75ICD, 9992. The purity of each fraction was demonstrated by probing for specific markers: splicing factor (1:100 dilution; Sigma Immunochemicals, St. Louis, Mo.) for the nuclear fraction; actin (1:200 dilution; Chemicon International Inc, Temecula, Calif.) for the membrane fraction; and Akt (1 μg/ml; Cell Signaling Technology Inc, Beverly, Mass.) for the cytosolic fraction.

Western Blot Analysis

Cell lysates or fractions were separated by SDS-PAGE and transferred to nitrocellulose membrane at 100V for 1 h. Membranes were blocked with 5% non-fat dried milk for 1 h and incubated with 1:2000 dilution of rabbit polyclonal anti-TACE antiserum or rabbit antisera against p75ECD [9651] or p75ICD [9992] overnight at 4° C., followed by 40 ng/ml donkey anti-rabbit IgG conjugated with HRP (Santa Cruz Biotechnology, Santa Cruz, Calif.) for 1 h at room temperature.

Reactions were developed using Super Signal West Pico Chemiluminescent substrate (Pierce Chemical Company, Rockford, Ill.). Membranes were reprobed for β-tubulin (1 μg/ml; Santa Cruz Biotechnology) to determine amounts of sample loaded. Bands were visualized with a photoimaging system using the shortest exposure time for development to allow visualization of major bands. Band densities were analyzed in Phosphorimager and statistical analyses were done using paired t-test.

Animals

Normal Sprague Dawley rats were maintained on a 12-hr light/dark cycle under broad-band fluorescent light (Sylvania Cool White, 45 foot-candles) in cages. To induce photochemical lesions, free-moving rats were exposed to long-wavelength blue light of 220 fc (Philips fluorescent lamps F40/BB) for 6 hrs after 24-hr dark adaptation. Animals were maintained in clear polycarbonate cages with minimal bedding during light exposure. Animals were allowed to recover in the dark for 24 hrs or 5 days after light exposure prior to sacrifice.

Under high dose of pentobarbital, the eyeballs were enucleated and processed for paraffin sectioning and immunohistochemistry. All experimental procedures were approved by the Institutional Animal Care and Use Committee of the University of North Texas Health Science Center in accordance with the guidelines outlined in the Guide for the Care and Use of Laboratory Animals (NIH Publication 85-23, revised 1996).

Immunohistochemistry.

Eyeballs were fixed in 4% paraformaldehyde in 0.1M sodium phosphate (pH 7.4) overnight, embedded in paraffin, and sectioned at 5 μm thickness. Paraffin sections were incubated in 0.1% Triton X-100 in PBS containing 0.05M glycine for 15 min and blocked with 5% normal goat serum, 5% BSA and 0.1% Triton X-100 for 1 h at room temperature. Sections were incubated with 1:200 dilution of rabbit polyclonal anti-TACE antiserum or rabbit polyclonal anti-p75NTR antisera, 9651 or 9992, in 3% normal goat serum and 1% BSA at 4° C. overnight. Sections were then incubated with 10 μg/ml anti-rabbit IgG conjugated to Alexa 488 (Molecular Probes Inc.) for TACE for 1 h, then counterstained with 300 nM DAPI (Molecular Probes Inc.) for 10 min. Images were photographed in Nikon microphot FXA microscope with epifluorescent attachment (Nikon Corp., Tokyo, Japan).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents

U.S. Pat. No. 6,509,355

Books
Collier, R J and Zigman, S, “Comparison of retinal photochemical lesions after exposure to Near-UV or short-wavelength visible radiation” In M M LaVail, R E Anderson and J G Hollyfield (Eds), Inherited and environmentally induced retinal degenerations, Alan R. Liss, Inc., New York (1989)

Other Publications
Abler, A S, Chang, C J, Fu, J and Tso, M, “Photic injury triggers apoptosis of photoreceptor cells” INV OPHTHALM & VIS SCI 35 (Suppl): 1517 (1994).
Abler et al., “Photic injury triggers apoptosis of photoreceptor cells,” RES. COMMUN. MOL. PATHOL. PHARMACOL. 92:177-189 (1996).
Al-Ubaidi, M R, Hollyfield, J G, Overbeek, P A and Baehr, W, “Photoreceptor degeneration induced by the expression of simian virus 40 large tumor antigen in the retina of transgenic mice” PROC NATL ACAD SCI USA 89(4): 1194-8 (1992).
Baker, S J and Reddy, E P, “Modulation of life and death by the TNF receptor superfamily,” ONCOGENE 17(25): 3261-70 (1998).
Barker, P A, Miller, F D, Large, T H and Murphy, R A, “Generation of the truncated form of the nerve growth factor receptor by rat Schwann cells. Evidence for post-translational processing” J BIOL CHEM 266(28):19113-9 (1991).
Barker, P A, “p75NTR: A study in contrasts” CELL DEATH DIFFER 5(5): 346-56 (1998).
Barrett, G L and Bartlett, P F, “The p75 nerve growth factor receptor mediates survival or death depending on the stage of sensory neuron development” PROC NATL ACAD SCI USA 91(14): 6501-5 (1994).
Black, R A, Rauch, C T, Kozlosky, C J, Peschon, J J, Slack, J L, Wolfson, M F, Castner, B J, Stocking, K L, Reddy, P, Srinivasan, S, Nelson, N, Boiani, N, Schooley, K A, Gerhart, M, Davis, R, Fitzner, J N, Johnson, R S, Paxton, R J, March, C J and Cerretti, D P, “A metalloproteinase disintegrin that releases tumour-necrosis factor-alpha from cells” NATURE 385(6618): 729-33 (1997).
Black, R A and White, J M, “ADAMs: focus on the protease domain” CURR OPIN CELL BIOL 10(5): 654-9 (1998).
Black, R A, “Tumor necrosis factor-alpha converting enzyme” INT J BIOCHEM CELL BIOL 34(1): 1-5 (2002).
Bressler et al., “Clinicopathologic correlation of drusen and retinal pigment epithelial abnormalities in age-related macular degeneration,” SUR OPHTHALM. 32(6):375-413 (1988).
Bronfman, F C, Tcherpakov, M, Jovin, T M and Fainzilber, M, “Ligand-induced internalization of the p75 neurotrophin receptor: a slow route to the signaling endosome” J NEUROSCI 23(8): 3209-3220 (2003).
Buchi, E R, “Cell death in the rat retina after a pressure-induced ischaemia-reperfusion insult: an electron microscopic study” EXP EYE RES 55: 605-613 (1992).
Buxbaum, J D, Liu, K N, Luo, Y, Slack, J L, Stocking, K L, Peschon, J J, Johnson, R S, Castner, B J, Cerretti, D P and Black, R A, “Evidence that tumor necrosis factor alpha converting enzyme is involved in regulated alpha-secretase cleavage of the Alzheimer amyloid protein precursor” J BIOL CHEM 273(43): 27765-7 (1998).
Casaccia-Bonnefil, P, Carter, B D, Dobrowsky, R T and Chao, M V, “Death of oligodendrocytes mediated by the interaction of nerve growth factor with its receptor p75” NATURE 383(6602): 716-9 (1996).
Chang, C J, Lai, W W, Edward, D P and Tso, M O “Apoptotic photoreceptor cell death after traumatic retinal detachment in humans” ARCH OPHTHALM 113: 880-886 (1995).
Collier, R, Waldron, W and Zigman, S, “Temporal sequence of changes to the ray squirrel retina after near-UV exposure” INV OPHTH & VIS SCI 30: 631-637 (1989).
Coulson, E J, Reid, K, Baca, M, Shipham, K A, Hulett, S M, Kilpatrick, T J and Bartlett, P F, “Chopper, a new death domain of the p75 neurotrophin receptor that mediates rapid neuronal cell death” J BIOL CHEM 275(39): 30537-45 (2000).
Cruikshanks, K J, Klein, R and Klein, E K, “Sunlight and age-related macular degeneration, The Beaver Dam Eye Study” ARCH OPHTH 111: 514-518 (1993).
Curcio et al., “Photoreceptor Loss in Age-Related Macular Degeneration,” IOVS 37(7):1236-1249 (1996).
DiStefano, P S, Chelsea, D M, Schick, C M and McKelvy, J F, “Involvement of a metalloprotease in low-affinity nerve growth factor receptor truncation: inhibition of truncation in vitro and in vivo” J NEUROSCI 13(6): 2405-14 (1993).
Edward et al., “Amelioration of Light-Induced Retinal Degeneration by a Calcium Overload Blocker,” ARCH. OPHTHALM. 109:554-622 (1991).
Fakforovich et al., “Photoreceptor degeneration in inherited retinal dystrophy delayed by basic fibroblast growth factor,” NATURE 347:83-86 (1990).
Frade, J M, Rodriguez-Tebar, A and Barde, Y A, “Induction of cell death by endogenous nerve growth factor through its p75 receptor” NATURE 383(6596): 166-8 (1996).
Frade, J M and Barde, Y A, “Genetic evidence for cell death mediated by nerve growth factor and the neurotrophin receptor p75 in the developing mouse retina and spinal cord” DEVELOPMENT 126(4): 683-90 (1999).
Fu et al., “Dexamethasone ameliorates retinal photic injury in albino rats,” EXP. EYE RES. 54:583-594 (1992).
Ge-Zhi et al., “Apoptosis in human retinal degenerations,” TRANS AM OPHTHALM SOC. 94:411-430 (1996).
Green, W R and Robertson, D M, “Pathologic fundings of photic retinopathy in the human eye” AM J OPHTH 112: 520-527 (1991).
Ham, W T, Jr., Ruffolo, J J, Jr, Mueller, H A and Guerry, D, III, “The nature of retinal radiation damage: dependence on wavelength, power level and exposure time” VISION RES 20: 1105-1111 (1980).
Harada, T, Harada, C, Nakayama, N, Okuyama, S, Yoshida, K, Kohsaka, S, Matsuda, H and Wada, K, “Modification of glial-neuronal cell interactions prevents photoreceptor apoptosis during light-induced retinal degeneration.” NEURON 26(2): 533-41 (2000).
Heldin, C-H and Ericsson, J, “Ripping tyrosine kinase receptors apart” SCIENCE 294: 2111-2112 (2001).
Heuer, J G, Fatemie-Nainie, S, Wheeler, E F and Bothwell, M, “Structure and developmental expression of the chicken NGF receptor” DEV BIOL 137(2): 287-304 (1990).
Heuer, J G, von Bartheld, C S, Kinoshita, Y, Evers, P C and Bothwell, M, “Alternating phases of FGF receptor and NGF receptor expression in the developing chicken nervous system” NEURON 5(3): 283-96 (1990).
Huber, L J and Chao, M V, “A potential interaction of p75 and trkA NGF receptors revealed by affinity crosslinking and immunoprecipitation.” J NEUROSCI RES 40(4):557-563 (1995).
Kahle, P and Hertel C, “Nerve growth factor (NGF) receptor on rat glial cell lines. Evidence for NGF internalization via p75NGFR” J BIOL CHEM 267: 13917-13923 (1992).
Kokaia, Z, Andsberg, G, Martinez-Serrano, A and Lindvall, O, “Focal cerebral ischemia in rats induces expression of p75 neurotrophin receptor in resistant striatal cholinergic neurons” NEUROSCIENCE 84: 1113-1125 (1998).
Kozaki et al., “Light-induced retinal damage in pigmented rabit: effect of alpha-tocopherol,” NIPPON GANKA GAKKAI ZASSHI 98(10):948-954 (1994).
Krishnamoorthy, R R, Crawford, M J, Chaturvedi, M M, Jain, S K, Aggarwal, B B, Al-Ubaidi, M R and Agarwal, N, “Photo-oxidative stress down-modulates the activity of nuclear.factor-kappaB via involvement of caspase-1, leading to apoptosis of photoreceptor cells” J BIOL CHEM 274(6): 3734-3743 (1999).
Kuwabara, T and Gom, R A, “Retinal damage by visible light. An electron microscopic study” ARCH OPHTHALMOL 79(1): 69-78 (1968).
Lam et al., “Amelioration of retinal photic injury in albino rats by dimethylthiourea,” ARCH. OPHTHALM. 108:1751-1757 (1990).
Lam et al., “Methylprednisolone therapy in laser injury of the retina,” GRAEFES ARCH. CLIN. EXP. OPHTHALM. 231:729-736 (1993).
LaVail, M M, “Survival of some photoreceptor cells in albino rats following long-term exposure to continuous light” INV OPHTH & VIS SCI 15: 64-70 (1976).
LaVail et al., “Multiple growth factors, cytokines, and neurotrophins rescue photoreceptors from the damaging effects of constant light,” PROC. NAT. ACAD. SCI. 89:11249-11253 (1992).
Lawwill, T, “Effects of prolonged exposure of rabbit retina to low-intensity light” INV OPHTH & VIS SCI 12: 45-51 (1973).
Lawwill, T, “Three major pathologic processes caused by light in the primate retina: a search for mechanisms” TRANS AM OPHTH SOC 80: 517-577 (1982).
Li et al., “Desferrioxamine ameliorates retinal photic injury in albino rats,” CUR. EYE RES. 10(2):133-144 (1991).
Li et al., “Amelioration of retinal photic injury by a combination of flunarizine and dimethylthiourea,” EXP. EYE RES. 56:71-78 (1993).
Liepinsh, E, Ilag, L L, Otting, G and Ibanez, C F, “NMR structure of the death domain of the p75 neurotrophin receptor” EMBO J. 16(16): 4999-5005 (1997).
Majdan, M, Lachance, C, Gloster, A, Aloyz, R, Zeindler, C, Bamji, S, Bhakar, A, Belliveau, D, Fawcett, J, Miller, F D and Barker, P A, “Transgenic mice expressing the intracellular domain of the p75 neurotrophin receptor undergo neuronal apoptosis” J NEUROSCI 17(18): 6988-98 (1997).
Markhall, J, Hamilton, A M and Bird, A C, “Histopathology of ruby and argon laser lesions in monkey and human retina” BRIT J OPHTH 59: 610-630 (1975).
Miller, F D and Kaplan, D R, “Life and death decisions: a biological role for the p75 neurotrophin receptor” CELL DEATH DIFFER 5(5): 343-5 (1998).
Moriya, M, Baker, B N and Williams, T P, “Progression and reversibility of early light-induced alterations in rat retinal rods” CELL TISSUE RES 246(3): 607-21 (1986).
Moss, M L, Jin, S L, Milla, M E, Bickett, D M, Burkhart, W, Carter, H L, Chen, W J, Clay, W C, Didsbury, J R, Hassler, D, Hoffnan, C R, Kost, T A, Lambert, M H, Leesnitzer, M A, McCauley, P, McGeehan, G, Mitchell, J, Moyer, M, Pahel, G, Rocque, W, Overton, L K, Schoenen, F, Seaton, T, Su, J L, Becherer, J D, “Cloning of a disintegrin metalloproteinase that processes precursor tumour-necrosis factor-alpha” NATURE 385(6618): 733-736 (1997).
Naash et al., “Induced acceleration of photoreceptor degeneration transgenic mice expressing mutant rhodopsin,” IOVS 37(5):775-782 (1996).
Noell, W K, Walker, V S, Kang, B S and Berman, S, “Retinal damage by light in rats” INVEST OPHTHALMOL 5(5): 450-473 (1966).
Oh, J D, Chartisathian, K, Chase, T N and Butcher, L L, “Overexpression of neurotrophin receptor p75 contributes to the excitotoxin-induced cholinergic neuronal death in rat basal forebrain” BRAIN RES 853: 174-185 (2000).
Organisciak et al., “Amelioration of photic injury in rat retina by ascorbic acid: a histopathologic study,” IOVS 26:1589-1598 (1985).
Organisckiak et al., “Protection by dimethylthiourea against retinal light damage in rats,” IOVS 33(5):1599-1609 (1992).
Organisciak, D T, Darrow, R M, Barsalou, L, Darrow, R A, Kutty, R K, Kutty, G and Wiggert, B, “Light history and age-related changes in retinal light damage” INVEST OPHTHALMOL VIS ScI 39(7): 1107-16 (1998).
Penn, J S, Tolman, B L, Thum, L A and Koutz, C A, “Effect of light history on the rat retina: time course of morphological adaptation and readaptation” NEUROCHEM RES 17(1): 91-9 (1992).
Peschon, J J, Slack, J L, Reddy, P, Stocking, K L, Sunnarborg, S W, Lee, D C, Russell, W E, Castner, B J, Johnson, R S, Fitzner, J N, Boyce, R W, Nelson, N, Kozlosky, C J, Wolfson, M F, Rauch, C T, Cerretti, D P, Paxton, R J, March, C J and Black, R A, “An essential role for ectodomain shedding in mammalian development” SCIENCE 282(5392): 1281-4 (1998).
Portera-Cailliau, C, Sung, C H, Nathans, J and Adler, R, “Apoptotic photoreceptor cell death in mouse models of retinitis pigmentosa” PNAS (usA) 91: 974-978 (1994).
Quigley, H A, Nickells, R W, Kerrigan, L A, Pease, M E, Thibault, D J and Zack, D J, “Retinal ganglion cell death in experimental glaucoma and after axotomy occurs by apoptosis” INV OPHHALM & VIS SCI 36: 774-786 (1995).
Rapp et al., “Evaluation of retinal susceptibility to light damage in pigmented rats supplemented with beta-carotene,” CUR. EYE RES. 15:219-223 (1995).
Roque, R S, Rosales, A A, Jingjing, L, Agarwal, N and Al-Ubaidi, M R, “Retina-derived microglial cells induce photoreceptor cell death in vitro” BRAIN RES 836(1-2): 110-119 (1999).
Rosales, A A and Roque, R S, “Microglia-derived cytotoxic factors part I: inhibition of tumor cell growth in vitro” BRAIN RES 748: 195-204 (1997).
Roux, P P and Barker, P A, “Neurotrophin signaling through the p75 neurotrophin receptor” PROG NEUROBIOL 67(3): 203-33 (2002).
Roux, P P, Colicos, M A, Barker, P A and Kennedy, T E, “p75 neurotrophin receptor expression is induced in apoptotic neurons after seizure” J NEUROSCI 19: 6887-6896 (1999).
Schlondorff, J and Blobel, C P, “Metalloprotease-disintegrins: modular proteins capable of promoting cell-cell interactions and triggering signals by protein-ectodomain shedding” J CELL SCI 112 (Pt 21): 3603-17 (1999).
Schlondorff, J, Becherer, J D and Blobel, C P, “Intracellular maturation and localization of the tumour necrosis factor alpha convertase (TACE)” BIOCHEM J 347: 131-138 (2000).
Shahinfar, S, Edward, D P and Tso, M O, “A pathalogic study of photoreceptor cell death in retinal photic injury” CURRENT EYE RES 10: 47-59 (1991).
Sheedlo, H J, Srinivasan, B, Brun-Zinkemagel, A M, Roque, C H, Lambert, W, Wordinger, R J and Roque, R S, “Expression of p75(NTR) in photoreceptor cells of dystrophic rat retinas” BRAIN RES MOL BRAIN RES 103(1-2): 71-9 (2002).
Sperling, H B, Johnson C and Harworth, R S, “Differential spectral photic damage to primate cones” VISION RES 20: 1117-1125 (1980).
Sykes, S M, Robison, W G, Jr, Waxler, M and Kuwabara, T, “Damage to the monkey retina by broad spectrum fluorescent light” INV OPHTH & VIS SCI 20: 425-434 (1981).
Taylor, H R, West, S, Munoz, B, Rosenthal, F S, Bressler, S B and Bressler, N M “The long term effects of visible light on the eye” ARCH OPHTH 110: 99-104 (1992).
Tso, M, “Photic maculopathy in rhesus monkey. Alight and electron microscopic study” INV OPHTH & VIS SCI 12: 17-34 (1973).
Vincent, B, Paitel, E, Saftig, P, Frobert, Y, Hartmann, D, De Strooper, B, Grassi, J, Lopez-Perez, E and Checler, F, “The disintegrins ADAM10 and TACE contribute to the constitutive and phorbol ester-regulated normal cleavage of the cellular prion protein” J BIOL CHEM 276(41): 37743-6 (2001).
Wang, X, Bauer, J H, Li, Y, Shao, Z, Zetoune, F S, Cattaneo, E and Vincenz, C “Characterization of a p75(NTR) apoptotic signaling pathway using a novel cellular model” J BIOL CHEM 276(36): 33812-20 (2001).
Young, R W, “Solar radiation and age-related macular degeneration” SURVEY OPHTH 32: 252-269 (1988).
Zhang, Y, Jiang J, Black R A, Baumann G and Frank S J, “Tumor necrosisfactor-a converting enzyme (TACE) is a growth hormone binding protein (GHBP) sheddase: the metalloprotease TACE/ADAM-17 is critical for (PMA-induced) GH receptor proteolysis a GHBP generation” ENDOCRINOLOGY 141(12): 4342-4348 (2000).
Zigman, S, Groff, J, Yulo, T and Vaughan, T, “The response of mouse ocular tissues to continuous near-UV light exposure” INV OPHTH & VIS SCI 14: 710-713 (1975).
Zupan, A A, Osborne, P A, Smith, C E, Siegel, N R, Leimgruber, R M and Johnson, E M, Jr., “Identification, purification, and characterization of truncated forms of the human nerve growth factor receptor” J BIOL CHEM 264(20): 11714-20 (1989).
Zupan, A A and Johnson, E M, Jr., “Evidence for endocytosis-dependent proteolysis in the generation of soluble truncated nerve growth factor receptors by A875 human melanoma cells” J BIOL CHEM 266(23): 15384-90 (1991).

1. A method of preventing neurodegeneration resulting from photic injury, said method comprising administering a therapeutically effective amount of a TACE inhibitor.

2. The method of claim 1, wherein said TACE inhibitor is IC-3.
